(19) United States
(12) Patent Application Publication
(10) Pub. No.: US 2018/0263824 A1
(43) Pub. Date: Sep. 20, 2018

(54) Compressive Bandage System and Method

(71) Applicant: Nicholas V. Ible, Coral Springs, FL (US)

(72) Inventor: Nicholas V. Ible, Coral Springs, FL (US)

(21) Appl. No.: 15/459,736

(22) Filed: Mar. 15, 2017

(51) Int. Cl. A61F 13/12; A61F 13/00

(52) U.S. Cl. CPC A61F 13/12; A61F 13/00029; A61F 2013/0028; A61F 13/00008; A61F 2013/00127; A61F 13/00038

(57) ABSTRACT

The compressive bandage includes an elastic cap to fit over the patient's head wound and head. The cap is impervious to fluid and has a fluid pervious inboard sleeve pocket. An absorbent pad placed in the sleeve since the sleeve and the cap expands. The loaded bandage is placed on the head wound. The pocket patch is a micro fiber, polyester fabric, preferably NIKE DRY FIT™ material. The head wound is stanched by placing the over the head wound. After the first use and after removal from the wounded head, the first pad is withdrawn from the pocket, a second pad in inserted and the compressive bandage is again placed on the head wound. After the first use or the second use, the bandage can be washed and sterilized.

The present invention relates to a compressive bandage system and a method for stanching a head wound.

### BACKGROUND OF THE INVENTION

There are various bandage systems utilized to cover and otherwise safeguard a wound. U.S. Pat. No. 8,163,973 to Johnson discloses an integrated wound dressing system or bandage, wherein a receptacle forms a pocket which contains a sterile wound treatment material, e.g. gauze. The flat pocket receptacle includes an inboard surface adapted to contact the wound. The bandage is wrapped around the limb or body and sometimes includes an occlusion layer. For example, the plastic layer is used as an occlusion layer which disposed adjacent to a non-adherent pad. The non-adherent pad is disposed directly on the wound. Alternatively, the plastic layer can be removed from the pocket receptacle and discarded such that no occlusion layer is used. In some embodiments, the plastic layer may be fixedly attached to the wound contacting surface such that when the dressing is applied, plastic layer abuts the wound and forms an occlusion layer.

U.S. Pat. No. 8,663,144 to Farrow et al. discloses a modular compression bandage device and method of assembly which provide for maximal access to a portion of a limb. These embodiments may have the compression band over the affected wound area on top, with the proximal and distal compression bands underlapping. This may allow removal of a compression band or bands over the affected area, while not requiring removal of the entire garment. Thus, the rest of the bandage garment may remain therapeutic while the area is accessed, and closures in the form of Velcro or hook and loop devices.

U.S. Pat. No. 8,764,694 to Daneshvar discloses a wound management system and methods with support zones for attachment means called “ATMs”. Sometimes the ATM is like VELCRO™. ATM2, on its upper surface which is made from hook fastener attachment means. ATM2B and ATM2S zones allow complementary pieces such as the body of the strap and a band and a gauze pad GP to be attached to them on a detachable, reattachable basis with a piece of gauze pad. This allows the gauze pad to be removed and replaced by a similar or another piece. This allows a soiled gauze pad to be exchanged with a fresh one.

U.S. Pat. No. 6,762,338 to Harder discloses a compression bandage with tightening means, wherein after the absorbent pad is located over the wound, the elastic roll is wrapped once around, and the roll is engaged into the free end of the S hook and tightened over the trauma by pulling on the elastic in the opposite direction of the initial wrap. The use of the Velcro™ strips allows ease of situating the pad and closure. The material is then passed through the S hook and pulled to provide increased tension in the material, thereby tightening the bandage over the wound and at the same time acting as a tourniquet to reduce blood loss.

U.S. Pat. No. 8,137,298 to Cureingtonsims discloses an absorbing medical binder bandage system and method which includes an absorbent pad surrounded by a lining. In one embodiment, lining may include a generally fluid impermeable material, such as thin plastic sheeting. In one embodiment, the lining also extends around a rear surface of inner layer such that it is positioned between inner layer and inner surface of outer layer during use. The lining located on a rear surface of inner layer and may inhibit absorbed fluids from contacting or otherwise leaking through onto outer layer or other portions of the surrounding environment. The lining is located around the edges of absorbent pad and may help to contain fluids in the absorbent pad such that they do not leak from edges of absorbent pad. In one embodiment, the lining around edges of absorbent pad contacts the skin of patient during use and/or inhibits edges of absorbent pad from contacting the skin of patient.

U.S. Pat. No. 6,512,159 to Shesol et al. discloses a facial wound dressing support device, including a flat pattern layout fullface mask that includes a first end, a second end, a top portion, a bottom portion and a center section. Also, the center section includes a nose opening and a mouth opening adapted to allow the patient to breathe and eat while wearing the fullface mask. In the bottom portion of the center section is a chin inverted “V” groove with an elongated hook fastener attached to one side of the groove.

U.S. Pat. No. 8,591,447 to Digrazia discloses a wound and bandage protection system and method with a panel with an external non-wound facing surface of the wound/bandage protector. A gauze pad may be affixed to the panel on an internal wound facing side. Alternatively, the gauze pad may be attached to the panel in a temporary fashion such as via use of a hook and loop type fastening system or a reusable pressure sensitive adhesive. In another alternative embodiment, the panel and the gauze pad is made of stretchable or super stretchable material. In this embodiment, the entire body may be configured to function as the panel.

U.S. Pat. No. 8,779,230 to Murphy et al. discloses a system and method for treating leg ulcers including an embodiment of a two layer compression bandage system wrapped around a patient's leg having one or more sores or ulcers to be treated, as well as one or more regions of healthy skin. The wound bandaging system comprises an inner foam layer and a separate, outer, elastic compression layer.

U.S. Pat. No. 9,375,346 to Sundheimer et al. discloses absorbent pads and related methods where the fluid permeable liner is configured to abut a fluid discharge area of a patient, whether human or animal. The fluid permeable liner forms the first side and second side of an absorbent pad, only on the first side of an absorbent pad, with the second side being formed by a fluid barrier, and the fluid barrier and fluid permeable liner joined together at their outer edges.

U.S. Pat. No. 9,271,877 to Mouton discloses a combined compression and absorption dressing/bandage where the wound dressing is formed by a rectangular laminate pad which comprises first and second absorbent layers and an inner layer sandwiched between and bonded to the first and second absorbent layers. The three layers are bonded together by a needle punching process.

U.S. Pat. No. 9,283,135 to Farrow discloses a compression sleeve augmenting calf muscle pump where the outer layer 112 may be a thin napped Lycra™ material to which a hook type fastener can readily adhere. The inner layer can be made of a compression stocking material for providing enhanced compression. The semi compressible layer may have channels sewn in them to create lines of natural lymph flow or crisscross pattern. Alternatively, in other embodiments the foam may serve as the inner layer with a loop compatible fabric orelastomeric material as the outer layer.

U.S. Pat. No. 7,118,545 to Boyde discloses wound dressing retainer and fastening device where wound dressing retainer includes a frame, a first flap, a second flap, a first raised structure, and a second raised structure. A frame may be of the same design, construction, and material as that of frame of wound dressing retainer, and may also be applied to wound dressing container. Absorbent dressing can be placed in frame and this dressing can be periodically replaced.

U.S. Pat. No. 6,049,022 to Tseng et al. discloses a gripping bandage where both inner strip and outer strip are cut in such a way that both strips have good stretchability in a lateral direction instead of in the longitudinal direction.

U.S. Pat. No. 6,258,051 to Shesol et al. discloses a disposable wound dressing and support unit that includes an elongated wrap having different lengths and widths depending on the application. The wrap has an outside and an inside. The inside of the wrap is placed against the skin of the patient. A gauze pad is also disclosed.

These prior art disclosures do not solve the problem of providing and easy to deploy compressive bandage with absorbent material and permitting that compressive bandage to be taken off the patient's head in order to replace an absorbent pad interposed between the head wound and the compressive outer bandage portion.

### OBJECTS OF THE INVENTION

It is an object of the present invention to provide a compressive bandage system to encase a portion of a patient's head (or substantially all of the patient's head, including the patient's head wound) and cover the patient's head wound.

It is another object of the present invention to provide a method of stanching a head wound on a patient's head.

It is a further object of the present invention to provide a compressive bandage system with a replaceable absorbent pad disposed between the outer elastic surface of the bandage and an inboard sleeve pocket in the compressive bandage.

It is another object of the present invention to utilize an inboard sleeve pocket that is pervious to fluid. The outer surface of the elastic cap being impervious to fluid.

It is an additional object of the present invention to provide a compressive bandage system that can be reused by washing (after the absorbent pads have been withdrawn from the inboard sleeve pocket).

### SUMMARY OF THE INVENTION

The compressive bandage system is adapted to compressively encase a patient's head wound with an elastic cap sized to fit over the patient's head over the patient's head and the wound itself. The cap is impervious to fluid. The cap has an inboard sleeve pocket formed by a pocket patch on an inboard surface segment of the cap. The pocket patch is pervious to fluid. A flat absorbent pad is disposed in the inboard sleeve pocket to form the compressive bandage. The inboard sleeve pocket expands permitting insertion of the flat pad due to elasticity of the inboard surface segment of the elastic cap prior to placement of the compressive bandage on the patient's head wound. In a preferred embodiment, the pocket patch is a micro fiber, polyester fabric adapted to transfer fluid away from the patient's head wound to the flat pad. Further, the pocket patch is made of NIKE DRY FIT™ material. The pocket patch is an elastic pocket patch. The flat pad preferably consists of gauze.

The method of stanching a head wound on a patient's head first provides an elastic cap sized to at least partially encase the patient's head about the head wound. Further the cap has an inboard sleeve pocket formed by a pocket patch on an inboard surface segment of the elastic cap. A flat absorbent pad is inserted into the inboard sleeve pocket to form a loaded compressive bandage. The loaded compressive bandage is placed over the head wound, substantially encasing the head wound. Fluid from the head wound passes through the pocket patch and accumulates in the flat pad while the bandage compresses the head wound with the loaded compressive bandage and thereby stanching the head wound. Fluid flow through the elastic cap is blocked due to the fluid impervious nature of the cap. Further, the healthcare worker can, after the loaded compressive bandage is deployed over the head wound, remove the loaded compressive bandage from the patient's head. The healthcare worker then withdraws the flat absorbent pad from the inboard sleeve pocket and inserts a second flat absorbent pad into the inboard sleeve pocket to form a second loaded compressive bandage. The worker places the second loaded compressive bandage over the head wound.

After the first use or the second use, the bandage can be washed and sterilized.

### BRIEF DESCRIPTION OF THE DRAWINGS

Further objects and advantages of the present invention can be found in the detailed descriptions of the preferred embodiments when taken in conjunction with the accompanying drawings in which:

FIG. 1 diagrammatically illustrates an exterior view of the compressive bandage system.

FIG. 2 diagrammatically illustrates a partial, cutaway view of the compressive bandage system with the flat absorbent pad disposed in the inboard sleeve pocket of the bandage.

FIG. 3 diagrammatically illustrates a cross-sectional view from the perspective of section line A′ to A″ FIG. 2.

FIG. 4 diagrammatically illustrates the method of stanching a head wound.

### DETAILED DESCRIPTION OF THE PREFERRED EMBODIMENTS

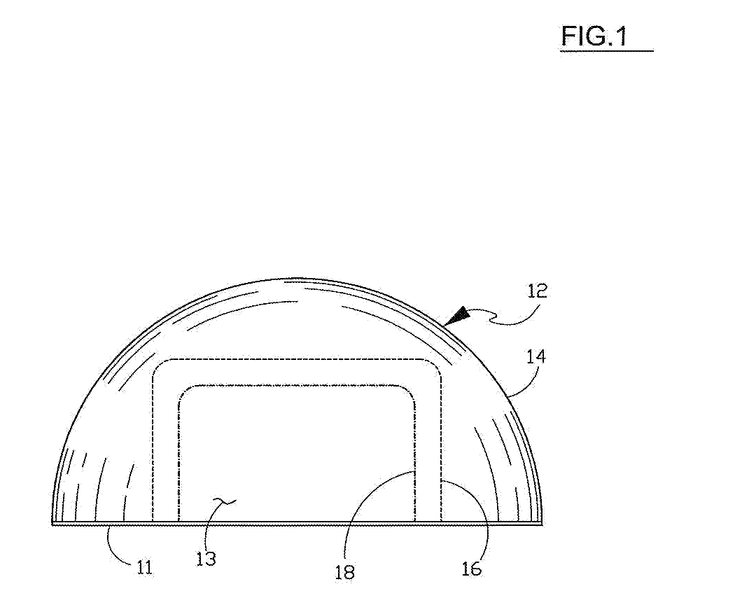

The present invention relates to a compressive bandage system and a method for stanching a head wound on a patient's head. Similar numerals designate similar item throughout the drawings. FIGS. 1,2 and 3 are concurrently discussed herein.

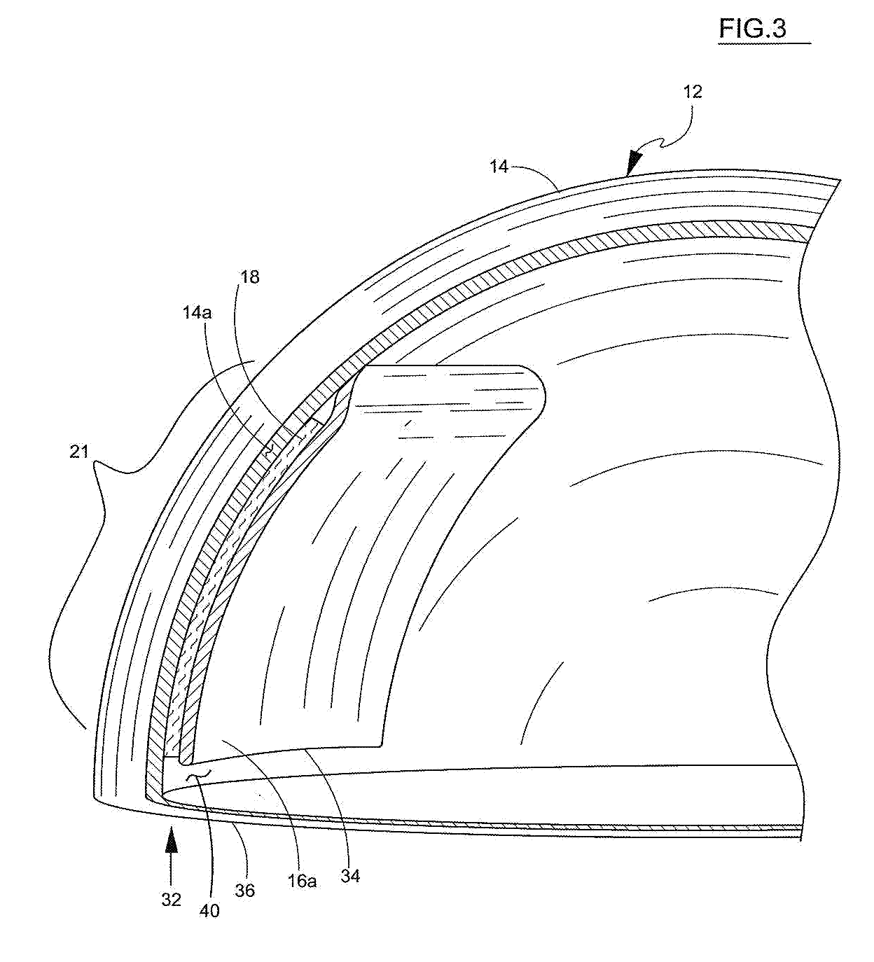

FIGS. 1, 2 and 3 diagrammatically show compressive bandage 12 formed as elastic cap sized to compressibility encase a portion of a patient's head and covering a patient's head wound. In a preferred embodiment, the outer shell of the cap is impervious to fluid. FIG. 1 diagrammatically shows an outer view of elastic cap 12 and, in dashed lines, inboard sleeve pocket 16 formed by pocket patch 16a shown in FIG. 3 as pocket patch 16a. The inboard sleeve pocket 16 in FIG. 1 is shown in dashed lines. In contrast, a flat absorbent pad 18 is shown in FIG. 1 as a dashed-long line-dashed symbol.

Inboard sleeve pocket 16 is formed on side 13 of elastic cap 14. The other side 11 of cap 14 (not shown in FIG. 1 except for the lower edge of cap side 11) in the preferred embodiment does not include an inboard sleeve pocket 16. However in a different embodiment, cap 14 may include an inboard sleeve pocket on opposite sides 11, 13 of elastic cap 14.

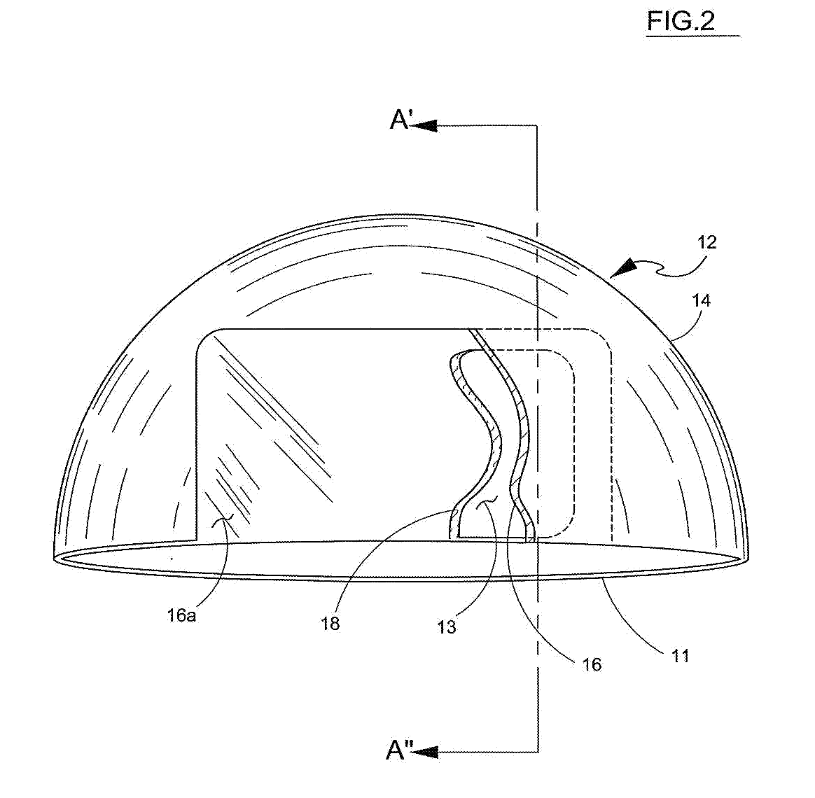

FIG. 2 diagrammatically illustrates a partial, cutaway view of the compressive bandage system 12. In FIG. 2, the outer surface portion 13 of elastic cap 14 is cutaway to reveal flat absorbent pad 18. In a preferred embodiment, absorbent pad 18 is made of gauze. The pocket patch 16a forms the inboard sleeve pocket 16.

In general, pocket patch 16 a is pervious to fluid. In a preferred embodiment, pocket patch 16a is made of a micro fiber, polyester fabric adapted to transfer fluid away from the patient's head wound to flat absorbent pad 18. Particularly, in the preferred embodiment pocket patch 16a is made of NIKE DRY FIT (trademark) material.

FIG. 3 is a cross-sectional view of the compressive bandage system 12 from the perspective of section line A′-A″ in FIG. 2. In FIG. 3, the absorbent pad 18 is slipped into and inserted into the sleeve 16 as shown by arrow 32. Due to the elastic nature of cap 14 and the elastic nature of pocket patch 16a, the health professional can easily open the inboard sleeve pocket 16 and insert flat absorbent pad 18 into the sleeve pocket. The lower edge 34 of sleeve pocket 16 may be raised above lower edge 36 of elastic cap 14 in order to provide a small tab permitting the healthcare professional to grab the small tab 40 to open inboard sleeve pocket 16. See tab segment 40 in FIG. 3. In a alternate embodiment, the lower edge 34 of inboard sleeve pocket 16 may extend outboard and outside the inboard portion of cap 14 beyond lower edge 36 of elastic cap 14. In this alternate embodiment, the tab surface 40 extends outboard or away from lower edge 36 of cap 14.

In operation, the inboard sleeve pocket 16 is flexible and is adapted to expand permitting insertion of the absorbent pad 18 due to the elasticity of the inboard surface segment of elastic cap 14 prior to placement of the compressive bandage 12 on the patient's head wound. Further, the pocket patch 16a is generally an elastic material.

After the compressive bandage system 12 is placed on the patient's head wound and the bandage stanches the head wound, the compressive bandage can be removed and the healthcare worker can withdraw the first installed pad 18 from inboard sleeve pocket 16. Thereafter, the healthcare worker can insert a second absorbent pad 18 into inboard sleeve pocket 16 and then place compressive bandage 14 on the head of the user.

Due to the materials used in the compressive bandage, after removal of the pad 18, the compressive bandage can be washed, sterilized and re-used.

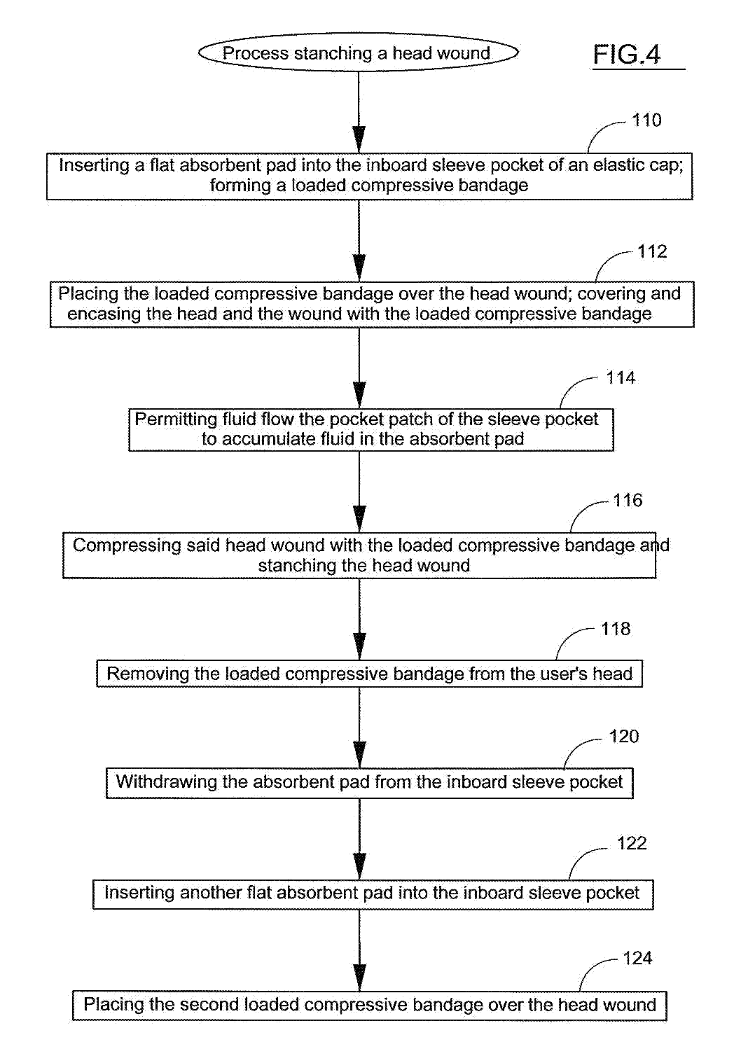

The method for stanching a head wound on a patient's head is shown as a process in FIG. 4. First, the healthcare worker inserts the absorbent pad into the inboard sleeve pocket 16 of the elastic cap (110). This forms a loaded compressive bandage as shown in the other figures and particularly shown in FIG. 3. Thereafter, the loaded compressive bandage is placed over the patient's head wound (112). In this manner, the elastic cap 14 encases a portion of or substantially all of the head of the patient as well as the patient's head wound.

Fluid flow from the head wound flows or passes through pervious pocket patch 16a (114) and into the sleeve pocket 16 and into the pad 18 and the fluid is accumulated and absorbed by absorbent pad 18 (116). The head wound is compressed by the loaded compressive bandage thereby stanching the head wound.

Thereafter, the healthcare worker removes the loaded compressive bandage 12 from the user's head (118). The healthcare worker withdraws the absorbent pad from the inboard sleeve pocket 16 (120). The healthcare worker inserts a second absorbent pad 18 into the inboard sleeve pocket 16 of the compressive bandage system (122). Thereafter the healthcare worker, using the second loaded compressive bandage (see FIG. 3), places the bandage over the patient's head wound (124).

The claims appended hereto are meant to cover modifications and changes within the scope and spirit of the present invention.

What is claimed is:

1. A compressive bandage system adapted to compressively encase a patient's head wound comprising:
an elastic cap sized to compressively encase a portion of said patient's head over said patient's head wound, said cap being impervious to fluid;
said cap having an inboard sleeve pocket formed by a pocket patch on an inboard surface segment of said elastic cap, said pocket patch being pervious to fluid; and
a flat absorbent pad disposed in said inboard sleeve pocket to form said compressive bandage; and
said inboard sleeve pocket adapted to expand permitting insertion of said flat pad due to elasticity of said inboard surface segment of said elastic cap prior to placement of said compressive bandage on said patient's head wound.

2. A compressive bandage system as claimed in claim 1 wherein said pocket patch is a micro fiber, polyester fabric adapted to transfer fluid away from said patient's head wound to said flat pad.

3. A compressive bandage system as claimed in claim 2 wherein said pocket patch is made of NIKE DRY FIT™ material.

4. A compressive bandage system as claimed in claim 1 wherein said pocket patch is an elastic pocket patch.

5. A compressive bandage system as claimed in claim 1 wherein said flat pad substantially consists of gauze.

6. A method for stanching a head wound on a patient's head comprising:
providing an elastic cap sized to at least partially encase said patient's head about said head wound;
providing an inboard sleeve pocket formed by a pocket patch on an inboard surface segment of said elastic cap;
inserting a flat absorbent pad into said inboard sleeve pocket to form a loaded compressive bandage;
placing said loaded compressive bandage over said head wound and substantially encasing said head wound with said loaded compressive bandage;
permitting fluid from said head wound to pass through said pocket patch and accumulate in said flat absorbent pad while compressing said head wound with said loaded compressive bandage and stanching said head wound therewith; and
prohibiting fluid flow through said elastic cap.

7. A method for stanching a head wound as claimed in claim 6 including:
removing said loaded compressive bandage from said patient's head;
withdrawing said flat absorbent pad from said inboard sleeve pocket;
inserting a second flat absorbent pad into said inboard sleeve pocket to form a second loaded compressive bandage; and
placing said second loaded compressive bandage over said head wound.

8. A method for stanching a head wound as claimed in claim 7 wherein after stanching said head wound with said second loaded compressive bandage, removing said second loaded compressive bandage from said patient's head; withdrawing said second flat absorbent pad from said inboard sleeve pocket and washing said elastic cap and inboard sleeve pocket.

9. A method for stanching a head wound as claimed in claim 5 wherein after stanching said head wound with said loaded compressive bandage, removing said loaded compressive bandage from said patient's head; withdrawing said flat absorbent pad from said inboard pocket sleeve and washing said elastic cap and said inboard pocket sleeve.

10. A method for stanching a head wound as claimed in claim 6 wherein said elastic cap prohibits fluid flow there through and wherein said pocket patch permits fluid flow there through.

11. A method for stanching a head wound as claimed in claim 9 wherein said elastic cap prohibits fluid flow there through and wherein said pocket patch permits fluid flow there through.

12. A method for stanching a head wound as claimed in claim 8 wherein said elastic cap prohibits fluid flow there through and wherein said pocket patch permits fluid flow there through.
